(19) United States
(12) Patent Application Publication
(10) Pub. No.: US 2024/0226504 A1
(43) Pub. Date: Jul. 11, 2024

(54) Adjustable Elongate Medical Device Handle

(71) Applicant: Bard Access Systems, Inc., Salt Lake City, UT (US)

(72) Inventors: Steffan Sowards, Salt Lake City, UT (US); William Robert McLaughlin, Bountiful, UT (US)

(21) Appl. No.: 18/095,448

(22) Filed: Jan. 10, 2023

(51) Int. Cl. A61M 25/01

(52) U.S. Cl. CPC A61M 25/0102; A61M 25/0136; A61M 2205/3306

(57) ABSTRACT

A catheter placement system includes a stylet disposed within a lumen of a catheter. The stylet includes an optical fiber configured for shape sensing. A device handle is selectively coupled with the stylet and selectively locked to the stylet to fix the position of the device handle along the stylet. A system module is operatively coupled with the stylet and logic of the system determines a shape of the stylet and depicts the shape on a display. The device handle is coupled with the stylet such that the optical fiber detects the position of the device handle along the stylet and the logic depicts a device handle icon together with the stylet shape. In accordance with temperature differences detected by the optical fiber, the logic determines the position of the insertion site along the stylet. The logic further depicts an insertion site icon together with the stylet shape.

### BACKGROUND

The placement of a catheter within a patient, such as a central catheter, requires careful manipulation of the catheter. It many instances, a stiffening stylet is inserted within a lumen of the catheter to assist the clinician in inserting the catheter. In some instances, a handle may be attached to the stylet to help the clinician grip the stylet. Guiding systems also help the clinician guide the catheter along the vasculature and further help the clinician position the distal tip of the stylet at the desired location, such as within the superior vena cava, for example. In some instances, a length of the catheter may be specifically defined, such as by trimming, so that a position of an external component of the catheter with respect to the insertion site may be indicative of the location of the distal tip of the catheter during and after placement of the catheter. In some instances, it may be advantageous to position the distal tip of the stylet adjacent the distal tip of the catheter. However, it may be difficult to maintain the position of the distal tip of the stylet adjacent the distal tip of the catheter during insertion. Furthermore, once mispositioned, it may be difficult to reposition the distal tip of the stylet adjacent the distal tip of the catheter after the insertion process is commenced. As such, it may be advantageous to monitor the position of the stylet with respect to the catheter via the guidance system. Furthermore, it may be advantageous to monitor the position of the stylet with respect to the insertion site.

Disclosed herein are catheter placement systems, devices, and methods that address the foregoing.

### SUMMARY

Disclosed herein is a catheter placement system that, according to some embodiments, includes an elongate medical device disposed within a lumen of a catheter, where the catheter is inserted within a vasculature of a patient. The elongate medical device includes a multi-core optical fiber extending along the elongate medical device, where the multi-core optical fiber includes a number of fiber-optic Bragg gratings disposed along the multi-core optical fiber, and where the fiber-optic Bragg gratings are configured to enable shape sensing of the elongate medical device. The system further includes (i) a device handle coupled with the elongate medical device at a location along the elongate medical device and (ii) a system module operatively coupled with the elongate medical device, where the system module includes a processor and memory having logic stored thereon that, when executed by the processor, performs operations of the system. The operations include determining a three-dimensional shape of the elongate medical device and depicting an image on a display of the system, where the image includes (i) the three-dimensional shape and a device handle icon at a position along the three-dimensional shape. In some embodiments, the fiber-optic Bragg gratings are configured to determine the location of the device handle along the elongate medical device.

In some embodiments, the device handle includes a locking mechanism configured for selective transitioning between an unlocked state, where the device handle is positionable along the elongate medical device and a locked state, where the location of the device handle along the elongate medical device is fixed.

In some embodiments, the device handle defines a positioning shape of the elongate medical device when the locking mechanism is transitioned to the locked state, and the operations include determining the location of the device handle along the elongate medical device based on the positioning shape.

In some embodiments, the device handle is rotatable about the elongate medical device in the unlocked state and device handle is rotatably fixed to the elongate medical device in the locked state.

In some embodiments, the operations include determining a rotational position of the device handle icon about the elongate medical device based on the positioning shape and the image includes an orientation of the device handle with respect to the three-dimension shape based on the rotational position of the device handle about the elongate medical device.

In some embodiments, the fiber-optic Bragg gratings are configured to enable sensing of a number of localized temperatures along the elongate medical device. The localized temperatures include a first localized temperature at a first point along the elongate medical device, and where the first point is located outside the patient adjacent an insertion site of the patient. The localized temperatures further include a second localized temperature at a second point along the elongate medical device, where the second point is located inside the patient adjacent the insertion site. In such embodiments, the operations include determining a location of the insertion site along the elongate medical device based on the first and second localized temperatures, and the image includes the insertion site icon at a position along the three-dimensional shape corresponding to the location of the insertion site along the elongate medical device.

Also disclosed herein is a medical device that, according to some embodiments, includes a device handle configured for selective attachment to an elongate medical device, where the device handle including a housing has a passageway extending through the housing, and where the passageway is configured to receive the elongate medical device therethrough. The elongate medical device includes a multi-core optical fiber extending along the elongate medical device, where the multi-core optical fiber includes a number of fiber-optic Bragg gratings disposed along the multi-core optical fiber, and where the device handle is configured to enable the fiber-optic Bragg gratings to detect a position of the device handle along the elongate medical device.

In some embodiments of the device, the housing is transitional between an open state and a closed state, where, in the closed state, the elongate medical device is laterally constrained within the passageway, and where, in the open state, the elongate medical device is laterally displaceable into and out of the passageway.

In some embodiments of the device, the device handle includes a locking mechanism configured for selective transitioning between an unlocked state, where the device handle is positionable along the elongate medical device and a locked state, where the location of the device handle is fixed along the elongate medical device.

In some embodiments of the device, transitioning the housing from the open state to the closed state transitions the locking mechanism from the unlocked state to the locked state.

In some embodiments of the device, the device handle is rotatable about the elongate medical device in the unlocked state, and the device handle is rotatably fixed to the elongate medical device in the locked state.

In some embodiments of the device, the device handle includes an actuator configured for manual actuation by a clinician, and the actuator is configured to transition the locking mechanism between the locked state and the unlocked state.

In some embodiments of the device, the locking mechanism includes an engagement surface configured to establish a frictional force between the engagement surface and the elongate medical device when the locking mechanism is transitioned to the locked state.

In some embodiments of the device, the locking mechanism includes a cam member that includes the engagement surface, where the cam member is rotatable between a first angular position and a second angular position. In such embodiments, the engagement surface is displaced away from the elongate medical device to define the unlocked state in the first angular position, and the engagement surface is disposed in frictional contact with the elongate medical device to define the locked state in the second angular position.

In some embodiments of the device, the housing is rotatable about the elongate medical device between a first orientation and a second orientation, where the second orientation is inverted in relation to the first orientation. In such embodiments, gravity causes the cam member to rotate toward the first angular position when the housing is disposed in the first orientation, and gravity causes the cam member to rotate toward the second angular position when the housing is disposed in the second orientation.

In some embodiments of the device, the locking mechanism forms a positioning shape of the elongate medical device when the locking mechanism is transitioned to the locked state, and the positioning shape enables the fiber-optic Bragg gratings to detect the position of the device handle along the elongate medical device.

Also disclosed herein is a method that, according to some embodiments, includes providing a stylet that includes a multi-core optical fiber extending along the stylet. The multi-core optical fiber includes a number of fiber-optic Bragg gratings disposed along the multi-core optical fiber, where the fiber-optic Bragg gratings are configured to enable shape sensing of the stylet, and where the stylet is operably coupled with a system module that (i) determines a three-dimensional shape of the stylet and (ii) depicts the three-dimensional shape on a display of the system module. The method further includes (i) inserting the stylet within a lumen of a catheter which in some embodiments includes positioning a distal end of the stylet adjacent a distal end of the catheter, (ii) coupling a stylet handle to the stylet, (iii) positioning the stylet handle adjacent an extension leg connector of the catheter, and (iv) locking the stylet handle to the stylet to fix the position of the stylet handle along the stylet, where locking the stylet handle forms a positioning shape of stylet. According to the method, the system module detects the positioning shape and depicts a handle icon on the display at a location along the three-dimensional shape consistent with the position of the stylet handle along the stylet. The method further includes inserting the stylet together with the catheter into the patient utilizing the stylet handle to manipulate the stylet.

In some embodiments, the method further includes visually monitoring the three-dimension shape in relation to the stylet handle icon to enable guiding the distal end of the stylet to a desired insertion location within the patient.

In some embodiments of the method, the fiber-optic Bragg gratings are configured to enable sensing of a number of localized temperatures along the stylet. The localized temperatures include (i) a first localized temperature at a first point along the stylet, where the first point is located outside the patient adjacent an insertion site of the patient, and (ii) a second localized temperature at a second point along the stylet, where the second point is located inside the patient adjacent the insertion site. In such embodiments, the system module determines a position of the insertion site along the stylet based on the first and second localized temperatures and depicts an insertion site icon on the display at a location along the three-dimensional shape, where the location along the three-dimensional shape is consistent with the position of the insertion site along the stylet. In such embodiments, the method further includes monitoring the location of the stylet handle icon with respect to the insertion site icon.

These and other features of the concepts provided herein will become more apparent to those of skill in the art in view of the accompanying drawings and following description, which describe particular embodiments of such concepts in greater detail.

### DRAWINGS

FIG. 1 illustrates a catheter placement system in use with a patient, in accordance with some embodiments.

FIG. 2 illustrates a detailed side view of an elongate medical device handle of the system of FIG. 1, in accordance with some embodiments.

FIG. 3 illustrates an end view of a second embodiment of the elongate medical device handle, in accordance with some embodiments.

FIG. 4A illustrates a cross-sectional side view of a third embodiment of the elongate medical device handle in an unlocked state, in accordance with some embodiments.

FIG. 4B illustrates a cross-sectional side view of the elongate medical device handle of FIG. 4A in a locked state, in accordance with some embodiments.

FIG. 5A illustrates a cross-sectional side view of a fourth embodiment of the elongate medical device handle in an unlocked state, in accordance with some embodiments.

FIG. 5B illustrates a cross-sectional side view of the elongate medical device handle of FIG. 5A in a locked state, in accordance with some embodiments.

FIG. 6 illustrates a flow chart of an exemplary method for placing a catheter within a patient, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The terms “proximal” and “distal” refer to opposite ends of a medical device, including the devices disclosed herein. As used herein, the distal end of a stylet is the end furthest from the practitioner during use, while the proximal end is the opposite end. For example, the distal end of stylet is defined as the end disposed within the patient. The proximal end is the end outside the patient.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

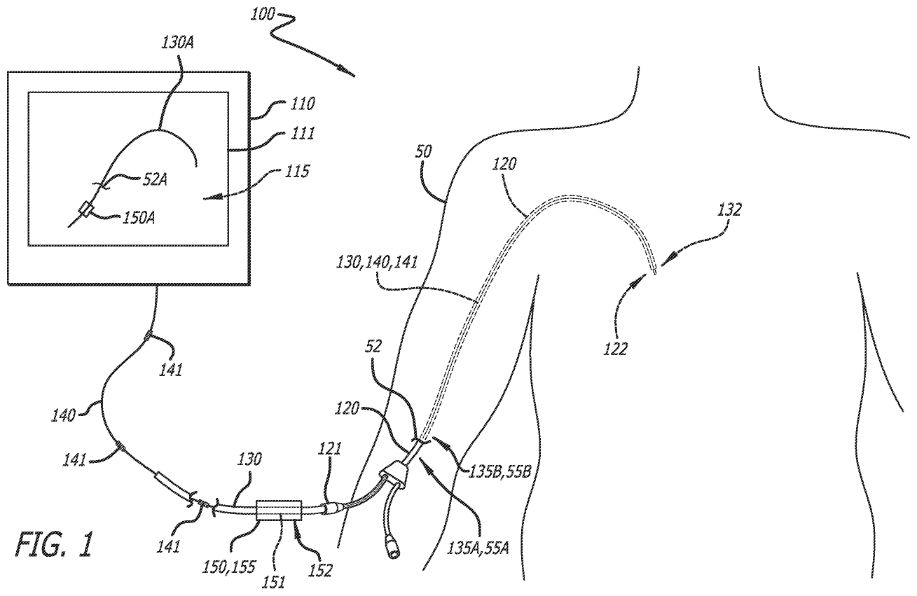

FIG. 1 illustrates the catheter placement system (system) 100 in use with a patient 50. A catheter 120 (e.g., a PICC in the illustrated instance) is advanced along a vasculature of the patient 50. An elongate medical device 130 is inserted into a lumen of the catheter 120 via an extension leg connector 121 of the catheter 120. In some embodiments, a distal end 132 of the elongate medical device 130 is disposed adjacent a distal end 122 of the catheter 120. The elongate medical device 130 includes an optical fiber 140 extending along the elongate medical device 130. The optical fiber 140 is operably coupled with a system module 110. The system module 110 includes a display 111 and a console 115.

The system 100 may (i) assist the clinician during advancement of the catheter 120 along the vasculature and (ii) assist the clinician during placement of the distal end 122 of the catheter 120 within the vasculature, such as within the superior vena cava, for example. The catheter 120 may include a central catheter, e.g., a central venous catheter (CVC) or a peripherally inserted center catheter (PICC), for example. The elongate medical device 130 may have sufficient length to extend between the distal end 122 of the catheter 120 and the extension leg connector 121 of the catheter 120. The elongate medical device 130 may include a stylet, a guidewire or any other elongate device suitable for insertion with a vasculature of the patient 50 and/or the lumen of the catheter 120.

The elongate medical device 130 includes an optical fiber 140 extending along the elongate medical device 130. The optical fiber 140 is a multi-core optical fiber including a number (e.g., 5, 10, 20, 50, 100 or more) of fiber-optic Bragg gratings 141 disposed along a length of the optical fiber 140. The optical fiber 140 is configured to enable shape sensing of the elongate medical device 130 based on reflected light signals emanating from (i.e., reflected by) the fiber-optic Bragg gratings 141. In the illustrated embodiment, the fiber-optic Bragg gratings 141 may be disposed along a length (e.g., an entire length) of the elongate medical device 130. A U.S. published patent application No. 2022-0034733 titled BRAGG GRATED FIBER-OPTIC FLUCTUATION SENSING AND MONITORING SYSTEM showing and describing a fiber-optic shape sensing system and method is included herein by reference in its entirety.

In some embodiments, the fiber-optic Bragg gratings 141 may be configured to detect conditions of the optical fiber 140, such as a temperature of the optical fiber 140, a motion of the optical fiber 140, or a displacement of fluid adjacent the optical fiber 140, such as via the Doppler effect, for example. In the illustrated embodiment, the optical fiber 140 extends away from the elongate medical device 130 so that the optical fiber 140 may be optically coupled with the system module 110, such as via an optical connection member (not shown), for example.

The system module 110 is operatively coupled with the elongate medical device 130. More specifically, the system module 110 is (i) optically coupled with the optical fiber 140. The system module 110 includes the console 115 having a processor and logic stored in memory (e.g., a non-transitory computer-readable medium). The logic, when executed by the processors, governs the operation of the system module 110. The console 115 further includes a light source and an optical receiver operably coupled with the optical fiber 140.

The system module 110 is configured to (i) determine, based on reflected light signals emanating the fiber-optic Bragg gratings 141, a three-dimensional shape 130A of the optical fiber 140, where the three-dimensional shape 130A represents the shape of the elongate medical device 130 and the catheter 120, and (ii) depict the three-dimensional shape 130A on the display 111. Said another way, the logic is configured to depict an image on the display 111, where the image includes at least the three-dimensional shape 130A. In some embodiments, the system module 110 may depict the three-dimensional shape 130A in real time during advancement of the catheter along the vasculature to assist the clinician in placement/guidance of the catheter 120.

The system 100 further includes a device handle (e.g., a stylet handle) 150 elongate medical device 130. The device handle 150 includes a passageway 151 and in use, the elongate medical device 130 is disposed within the passageway 151. In some instances of use, the device handle 150 may be coupled with the elongate medical device 130 adjacent the extension leg connector 121. In some embodiments, the device handle 150 may be configured for slidable displacement along the elongate medical device 130. In further embodiments, device handle 150 may be frictionally coupled with the elongate medical device 130 such that a longitudinal position of the device handle 150 is generally fixed in the absence of a deliberate displacement action by a clinician.

In some embodiments, the fiber-optic Bragg gratings 141 are configured to enable sensing of a number of localized temperatures along the elongate medical device 130. For example, the fiber-optic Bragg gratings 141 may enable sensing of a first localized temperature 55A (e.g., a room temperature) at a first point 135A along the elongate medical device 130, where the first point 135A is located outside the patient 50 adjacent an insertion site 52 of the patient 50. Further, the fiber-optic Bragg gratings 141 may enable sensing of a second localized temperature 55B (e.g., a body temperature) at a second point 135B along the elongate medical device 130, where the second point 135B is located inside the patient 50 adjacent an insertion site 52 of the patient 50. As such, the logic may determine a location of the insertion site 52 along the elongate medical device 130. Having determined the location of the insertion site 52, the logic may include an insertion site icon 52 within the image at position along the three-dimensional shape 130A corresponding to the location of the insertion site along the elongate medical device 130. In some embodiments, the system module 110 may depict the insertion site icon 52 along the three-dimensional shape 130A in real time during advancement of the catheter along the vasculature to assist the clinician in placement/guidance of the catheter 120. As such, the clinician may monitor the position of the insertion site icon 52A along the three-dimensional shape 130A during advancement of the stylet 130 along the vasculature.

In the illustrated embodiment, the device handle 150 may include a locking mechanism 155 configured for selective transitioning between an unlocked state and a locked state. In the unlocked state, the device handle 150 may be displaced along the elongate medical device 130. Conversely, in the locked state, the device handle 150 may be attached to the elongate medical device 130 such that the longitudinal position is fixed, i.e., the device handle 150 generally immovable along the elongate medical device 130. As such, the clinician may manipulate the device handle 150 to longitudinally displace the elongate medical device 130.

In some embodiments, the device handle 150 is rotatable about the elongate medical device 130 in the unlocked state and device handle 150 is rotatably fixed to the elongate medical device 130 in the locked state. In such embodiments, the clinician may manipulate the device handle 150 to rotate the elongate medical device 130 during use.

The device handle 150 includes a housing 152 which generally defines the structure of the device handle 150. The housing 152 may be formed of any suitable material sufficiently rigid to enable the functionality of the device handle 150. In some embodiments, the housing 152 may be formed of a plastic material, such as a thermoplastic material, for example. In some embodiments, the housing 152 may be formed via a plastic injection molding process.

The housing 152 may include a geometry to assist the clinician in gripping the device handle 150. In some embodiments, the housing 152 may include finger depressions and/or raised portions. In some embodiments, the housing 152 may include dimples, protrusions, ribs, grooves, a rough surface, or any combination thereof. In some embodiments, the geometry of the housing 152 may be symmetrical. In some embodiments, the geometry may indicate an orientation of the device handle 150. For example, one side of the housing 152 may include features that indicate that the one side is a top side of the device handle 150.

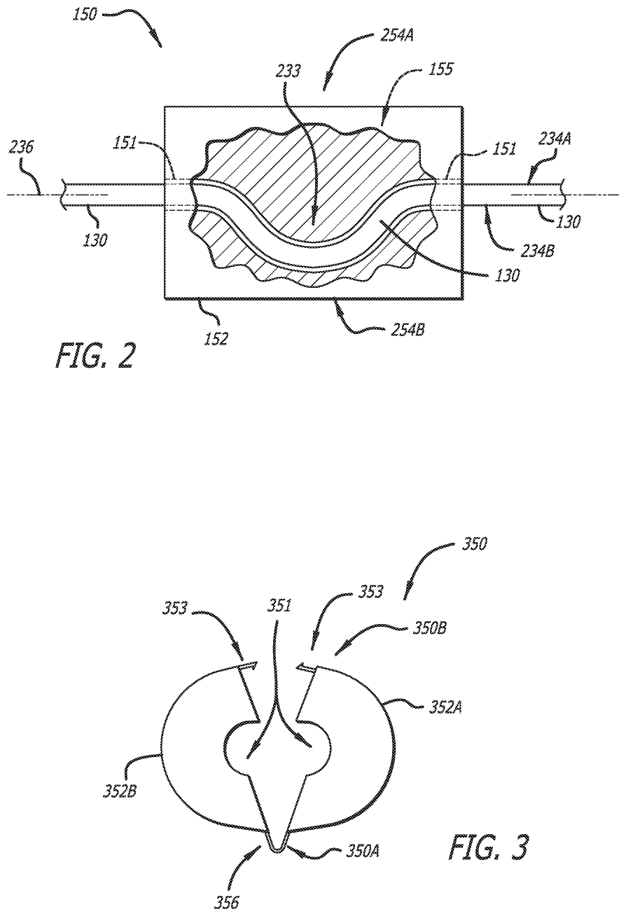

FIG. 2 illustrates a cut-away detailed view of the device handle 150 including the elongate medical device 130 extending through the passageway 151. The locking mechanism 155 may be configured to define a positioning shape 233 of the elongate medical device 130 within the device handle 150. The fiber-optic Bragg gratings 141 enable sensing of the positioning shape 233. The logic may recognize the positioning shape 233 apart from other three dimensional shapes of the elongate medical device 130 during use. As such, the logic may determine that the device handle 150 is locked onto the elongate medical device 130 at the location of the positioning shape 233 along the elongate medical device 130. Having determined the location of the positioning shape 233, the logic may depict a device-handle icon 150A at a position along the three-dimension shape 130A consistent with the location of the positioning shape 233 along the elongate medical device 130.

In some embodiments, the logic may determine a rotational orientation of the elongate medical device 130 about a longitudinal axis 236 thereof based on the positioning shape 233. For example, the logic may relate a first lateral side 234A of the elongate medical device 130 with a first side 254A of the device handle 150. Similarly, the logic may relate a second lateral side 234B of the elongate medical device 130 with a second side 254B of the device handle 150, where the second lateral side 234B is opposite the first lateral side 234A, and where the second side 254B is opposite the first side 254A. Although not shown, the device-handle icon 150A may include the first and second sides 254A, 254B so that the device-handle icon 150A indicates the rotational orientation of the three-dimensional shape 130A.

It is noted that the positioning shape 233 is just one exemplary mechanism to enable the fiber-optic Bragg gratings 141 to sense the location of the device handle 150 along the elongate medical device 130. Other mechanisms may be employed to enable the fiber-optic Bragg gratings 141 to sense the location of the device handle 150 along the elongate medical device 130, such a light reflecting surface, for example. As such, any and all other mechanisms as may be contemplated by one of ordinary skill are included in this disclosure.

FIG. 3 is an end view illustration of second embodiment of a device handle 350 that may resemble certain features and functionalities of the device handle 150. The device handle 350 includes a first housing half 352A and a second housing half 352B coupled together via a hinge 356. The hinge 356 enables the device handle 350 to transition between an open state (shown) and a closed state. The device handle 350 may include a latch 353 configured to secure the device handle 350 in the closed state. In the open state, the device handle 350 allows for lateral disposition of the elongate medical device 130 (FIG. 1) into and out of the passageway 351. In the closed state, the device handle 350 prevents lateral removal of the elongate medical device 130 from the passageway 351.

In some embodiments, the transitioning the device handle 350 toward the closed state, having the elongate medical device 130 disposed within the passageway 351, may define the device handle 350 in the unlocked state, i.e., the device handle 350 may be slidably displaced along the elongate medical device 130 when the device handle 350 is transitioned to the closed state. In other embodiments, the transitioning the device handle 350 toward the closed state, having the elongate medical device 130 disposed within the passageway 351, may define the device handle 350 in the locked state, i.e., slidable displacement of the device handle 350 along the elongate medical device 130 when the device handle 350 is transitioned to the closed state may be prevented. In some embodiments, transitioning the device handle 350 toward the closed state, having the elongate medical device 130 disposed within the passageway 351, may be define the positioning shape 233 (see FIG. 2) of the elongate medical device 130.

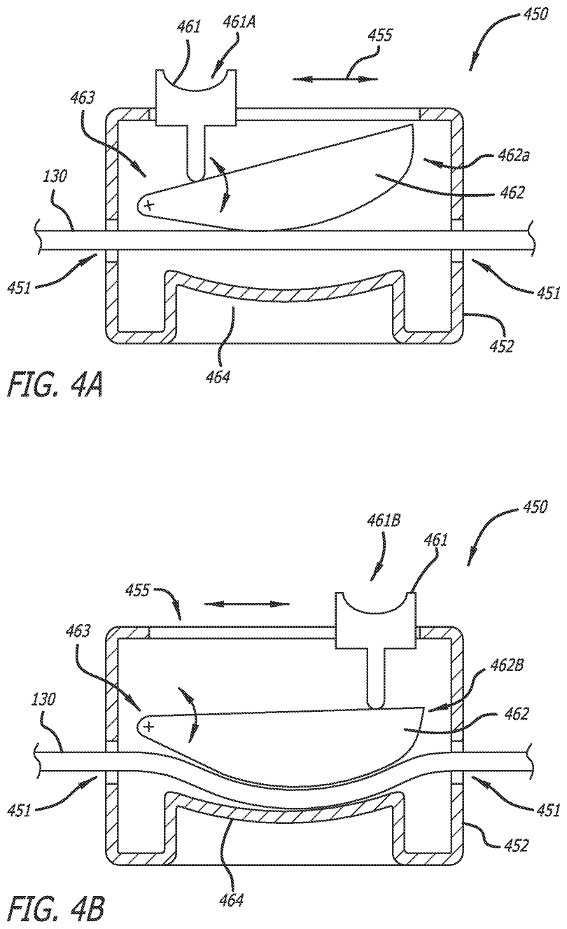

FIGS. 4A-4B illustrate a third embodiment of a device handle 450 that may resemble certain features and functionalities of the device handle 150. An exemplary locking mechanism 455 is generally configured to transition between the unlocked state and the locked state as a result of manipulation by the clinician. Shown are the housing 452 (in cross section) and the passageway 451 with the elongate medical device 130 passing therethrough. FIG. 4A shows the locking mechanism 455 in the unlocked state, and FIG. 4B shows the locking mechanism 455 in the locked state. The locking mechanism 455 includes an actuator 461 and a cam member 462. The actuator 461 is positionable by the clinician between a unlock position 461A and lock position 461B. The cam member 462 is pivotable about a pivot point 463 between non-clamping position 462A and a clamping position 462B. In the clamping position 462B, the cam member 462 sandwiches the elongate medical device 130 between the cam member 462 and a wall 464 of the housing 452 to define a friction force between the device handle 450 and the elongate medical device 130. The friction force prevents longitudinal displacement and/or rotation of the device handle 450 with respect to the elongate medical device 130. In some embodiments, the sandwiching the elongate medical device 130 between the cam member 462 and a wall 464 defines a positioning shape 433 of the elongate medical device 130. The actuator 461 engages the cam member 462 such that (i) when the actuator 461 is in the unlock position 461A, the cam member 462 is disposed in the non-clamping position 462A and (ii) when the actuator 461 is in the lock position 461B, the cam member 462 is pivoted to the clamping position 462B.

In use, the clinician may displace the device handle 450 along the elongate medical device 130 to a desired location. Thereafter, the clinician may slide the actuator 461 away from the unlock position 461A to the lock position 461B to fix the location of the device handle 450 on the elongate medical device 130. In some instances, the clinician may slide the actuator away from the lock position 461B to the unlock position 461A to adjust the location of the device handle 450 on the elongate medical device 130. Thereafter, the clinician may slide the actuator away from the unlock position 461A to the lock position 461B to fix the location of the device handle 450 on the elongate medical device 130 once again.

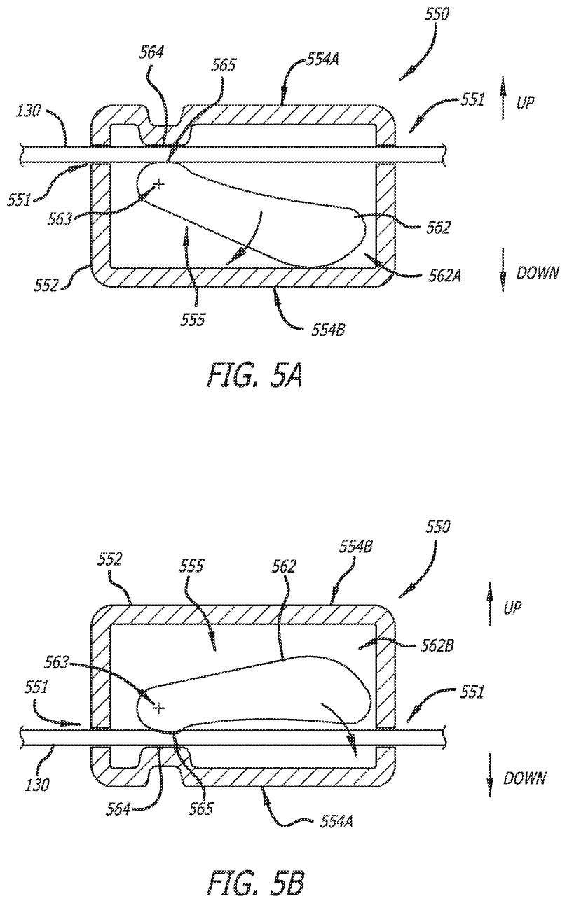

FIGS. 5A-5B illustrate a fourth embodiment of a device handle 550 that may resemble certain features and functionalities of the device handle 150. Another exemplary locking mechanism 555 is generally configured to transition between the unlocked state and the locked state as a result of rotation of the device handle 550 by the clinician about the elongate medical device 130. Shown are the housing 552 (in cross section) and the passageway 551 with the elongate medical device 130 passing therethrough. FIG. 5A shows the locking mechanism 555 in the unlocked state, and FIG. 5B shows the locking mechanism 555 in the locked state. The locking mechanism 555 includes a gravity actuated cam member 562 that is pivotable about a pivot point 563. In the unlocked state of FIG. 5A, the device handle 450 is disposed in a first orientation where a first side 554A is disposed upward and a second side 554B is disposed downward. In the first orientation, gravity causes the cam member 562 to rotate toward a non-clamping position 562A, where a cam surface 565 is displaced away from the elongate medical device 130. Similarly, in the locked state of FIG. 5B, the device handle 450 is disposed in a second orientation (inverted with respect to the first orientation) where the first side 554A is disposed downward and the second side 554B is disposed upward. In the second orientation, gravity causes the cam member 562 to rotate toward the clamping position 562B, where the cam member 562 sandwiches the elongate medical device 130 between the cam surface 565 and the wall 564 to define the friction force between the device handle 550 and the elongate medical device 130. The friction force prevents longitudinal displacement and/or rotation of the device handle 550 with respect to the elongate medical device 130. In some embodiments, the sandwiching the elongate medical device 130 between the cam surface 565 and the wall 564 may define a positioning shape (not shown, but see FIG. 2) of the elongate medical device 130.

In use, the clinician may rotate the device handle 550 to the first orientation to enable the device handle 550 to slide along the elongate medical device 130 to a desired location. Thereafter, the clinician may rotate the device handle 550 to the second orientation to fix the location of the device handle 550 on the elongate medical device 130.

It is noted that the locking mechanisms 455 and 555 are just two exemplary locking mechanisms of many locking mechanisms that may be contemplated by one of ordinary skill. As such, any and all other locking mechanisms suitable for selectively locking and unlocking the device handle 150 to and from the elongate medical device 130, as may be contemplated one of ordinary skill, are included in this disclosure.

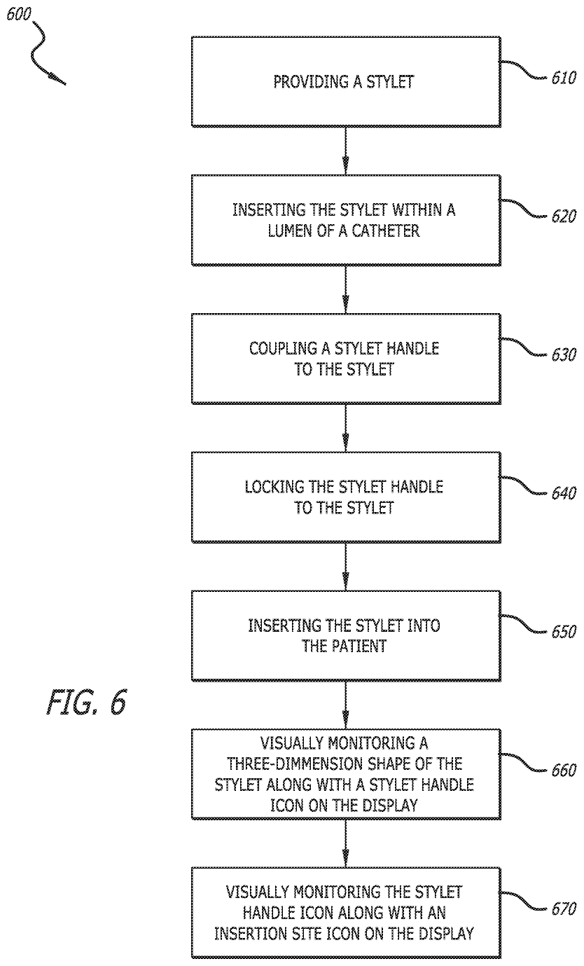

FIG. 6 illustrates a flow chart of an exemplary method for placing a central catheter within a patient that may include all or any subset of the following steps, actions, or processes. The method 600 may include providing a stylet (block 610). The stylet includes a multi-core optical fiber having a number of fiber-optic Bragg gratings disposed along the multi-core optical fiber, where the fiber-optic Bragg gratings are configured to enable shape sensing of the stylet. The stylet is operably coupled with a system module that (i) determines a three-dimensional shape of the stylet and (ii) depicts the three-dimensional shape on a display of the system module. The method 600 may further include inserting the stylet within a lumen of a catheter (block 620). In some embodiments, inserting the stylet may include positioning a distal end of the stylet adjacent a distal end of the catheter. The method 600 may further include coupling a stylet handle to the stylet (block 630). In some embodiments, coupling a stylet handle to the stylet includes positioning the stylet handle adjacent an extension leg connector of the catheter. The method 600 may further include locking the stylet handle to the stylet (block 640) to fix the position of the stylet handle along the stylet. In some embodiments, locking the stylet handle to the stylet forms a positioning shape of stylet. According to such embodiments, the system module detects the positioning shape and depicts a handle icon on the display at a location along the three-dimensional shape consistent with the position of the stylet handle along the stylet. The method 600 may further include inserting the stylet into the patient (block 650). In some embodiments, the stylet is inserted together with the catheter into the patient utilizing the stylet handle to manipulate the stylet.

The method 600 may further include visually monitoring the three-dimension shape on the display (block 660) to enable guiding the distal end of the stylet to a desired insertion location within the patient. In some embodiments, the three-dimension shape is specifically monitored in relation to the handle icon.

In some embodiments of the method 600, the fiber-optic Bragg gratings are configured to enable sensing of a number of localized temperatures along the stylet. The localized temperatures include (i) a first localized temperature at a first point along the stylet, where the first point is located outside the patient adjacent an insertion site of the patient, and (ii) a second localized temperature at a second point along the stylet, where the second point is located inside the patient adjacent the insertion site. In such embodiments, the system module determines a position of the insertion site along the stylet based on the first and second localized temperatures and depicts an insertion site icon on the display at a location along the three-dimensional shape, where the location along the three-dimensional shape is consistent with the position of the insertion site along the stylet. In such embodiments, the method 600 may further includes visually monitoring the location of the location of the stylet handle icon with respect to the insertion site icon (block 670).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter placement system, comprising:
an elongate medical device disposed within a lumen of a catheter inserted within a vasculature of a patient, wherein:
the elongate medical device includes a multi-core optical fiber extending along the elongate medical device,
the multi-core optical fiber includes a number of fiber-optic Bragg gratings disposed along the multi-core optical fiber, and
the fiber-optic Bragg gratings are configured to enable shape sensing of the elongate medical device;
a device handle coupled with the elongate medical device at a location along the elongate medical device; and
a system module operatively coupled with the elongate medical device, the system module including a processor and memory having logic stored thereon that, when executed by the processor, performs operations that include:
determining a three-dimensional shape of the elongate medical device; and
depicting an image on a display of the system, wherein the image includes:
the three-dimensional shape; and
a device handle icon at a position along the three-dimensional shape.

2. The system according to claim 1, wherein the fiber-optic Bragg gratings are configured to determine the location of the device handle along the elongate medical device.

3. The system according to claim 1, wherein the device handle includes a locking mechanism configured for selective transitioning between:
an unlocked state, wherein the device handle is positionable along the elongate medical device, and
a locked state, wherein the location of the device handle along the elongate medical device is fixed.

4. The system according to claim 3, wherein:
the device handle defines a positioning shape of the elongate medical device when the locking mechanism is transitioned to the locked state, and
the operations include determining the location of the device handle along the elongate medical device based on the positioning shape.

5. The system according to claim 3, wherein:
in the unlocked state the device handle is rotatable about the elongate medical device, and
in the locked state the device handle is rotatably fixed to the elongate medical device.

6. The system according to claim 5, wherein:
the operations include determining a rotational position of the device handle about a longitudinal axis of the elongate medical device based on the positioning shape, and
the image includes an orientation of the device handle icon with respect to the three-dimension shape based on the rotational position of the device handle about the longitudinal axis of the elongate medical device.

7. The system according to claim 1, wherein:
the fiber-optic Bragg gratings are configured to enable sensing of a number of localized temperatures along the elongate medical device, including:
a first localized temperature at a first point along the elongate medical device, the first point located outside the patient adjacent an insertion site of the patient; and
a second localized temperature at a second point along the elongate medical device, the second point located inside the patient adjacent the insertion site,
the operations include determining a location of the insertion site along the elongate medical device based on the first and second localized temperatures, and
the image includes the insertion site at a position along the three-dimensional shape corresponding to the location of the insertion site along the elongate medical device.

8. A medical device, comprising:
a device handle configured for selective attachment to an elongate medical device, the device handle including a housing having a passageway extending through the housing, the passageway configured to receive the elongate medical device therethrough,
wherein:
the elongate medical device includes a multi-core optical fiber extending along the elongate medical device,
the multi-core optical fiber includes a number of fiber-optic Bragg gratings disposed along the multi-core optical fiber, and
the device handle is configured to enable the fiber-optic Bragg gratings to detect a position of the device handle along the elongate medical device.

9. The device according to claim 8, wherein the housing is transitional between an open state and a closed state, wherein:
in the closed state, the elongate medical device is laterally constrained within the passageway, and
in the open state, the elongate medical device is laterally displaceable into and out of the passageway.

10. The device according to claim 8, wherein the device handle includes a locking mechanism configured for selective transitioning between:
an unlocked state, wherein the device handle is positionable along the elongate medical device, and
a locked state, wherein the location of the device handle is fixed along the elongate medical device.

11. The device according to claim 10, wherein transitioning the housing from the open state to the closed state transitions the locking mechanism from the unlocked state to the locked state.

12. The device according to claim 10, wherein:
the device handle is rotatable about the elongate medical device in the unlocked state, and
the device handle is rotatably fixed to the elongate medical device in the locked state.

13. The device according to claim 10, wherein:
the device handle includes an actuator configured for manual actuation by a clinician, and
the actuator is configured to transition the locking mechanism between the locked state and the unlocked state.

14. The device according to claim 10, wherein the locking mechanism includes an engagement surface configured to establish a frictional force between the engagement surface and the elongate medical device when the locking mechanism is transitioned to the locked state.

15. The device according to claim 14, wherein the locking mechanism includes a cam member that includes the engagement surface, the cam member rotatable between a first angular position and a second angular position, wherein:
in the first angular position, the engagement surface is displaced away from the elongate medical device to define the unlocked state, and
in the second angular position, the engagement surface is disposed in frictional contact with the elongate medical device to define the locked state.

16. The device according to claim 15, wherein:
the housing is rotatable about the elongate medical device between a first orientation and a second orientation inverted in relation to the first orientation,
in the first orientation, gravity causes the cam member to rotate toward the first angular position, and
in the second orientation, gravity causes the cam member to rotate toward the second angular position.

17. The device according to claim 10, wherein:
the locking mechanism forms a positioning shape of the elongate medical device when the locking mechanism is transitioned to the locked state, and
the positioning shape enables the fiber-optic Bragg gratings to identify the position of the device handle along the elongate medical device.

18. A method of placing a catheter within a patient, comprising:
providing a stylet, comprising a multi-core optical fiber having a number of fiber-optic Bragg gratings disposed along the multi-core optical fiber, the fiber-optic Bragg gratings configured to enable shape sensing of the stylet, wherein the stylet is operably coupled with a system module that (i) determines a three-dimensional shape of the stylet and (ii) depicts the three-dimensional shape on a display of the system module;
inserting the stylet within a lumen of a catheter;
coupling a stylet handle to the stylet, the stylet handle configured to enable manipulation of the stylet;
positioning the stylet handle adjacent an extension leg connector of the catheter;
locking the stylet handle to the stylet to fix the position of the stylet handle along the stylet, wherein:
locking the stylet handle forms a positioning shape of stylet, and
the system module (i) detects the positioning shape and (ii) depicts a stylet handle icon on the display at a location along the three-dimensional shape consistent with the position of the stylet handle along the stylet; and
inserting the stylet together with the catheter into the patient utilizing the stylet handle.

19. The method according to claim 18, further comprising visually monitoring the three-dimension shape in relation to the stylet handle icon to enable guiding the distal end of the stylet to a desired insertion location within the patient.

20. The method according to claim 19, wherein:
the fiber-optic Bragg gratings are configured to enable sensing of a number of localized temperatures along the stylet, including:
a first localized temperature at a first point along the stylet, the first point located outside the patient adjacent an insertion site of the patient; and
a second localized temperature at a second point along the stylet, the second point located inside the patient adjacent the insertion site,
the system module (i) determines a position of the insertion site along the stylet based on the first and second localized temperatures and (ii) depicts an insertion site icon on the display at a location along the three-dimensional shape consistent with the position of the insertion site along the stylet, and
the method further comprises monitoring the location of the stylet handle icon with respect to the insertion site icon.
